(19) United States
(12) Patent Application Publication
(10) Pub. No.: US 2010/0326575 A1
(43) Pub. Date: Dec. 30, 2010

(54) Synthesis of 2-nitroimino-5-nitrohexahydro-1,3,5-triazine

(76) Inventor: Cory G. Miller, Rochester, MI (US)

Correspondence Address: L.C. BEGIN & ASSOCIATES, PLLC, 510 HIGHLAND AVENUE, PMB 403, MILFORD, MI, 48381, US

(21) Appl. No.: 11/699,712

(22) Filed: Jan. 29, 2007

Related U.S. Application Data
(60) Provisional application No. 60/762,839, filed on Jan. 27, 2006.

(51) Int. Cl. C07D 251/48; C06B 31/38; C06B 31/14; C06B 29/00; C06B 29/22; C06B 29/16; C06B 25/34

(52) U.S. Cl. 149/55; 149/67; 149/75; 149/76; 149/78; 149/92; 544/204

(57) ABSTRACT

A method of forming 2-nitroimino-5-nitrohexahydro-1,3,5-triazine is presented. The method contains the steps of mixing nitroguanidine, formaldehyde, tert-butyl amine, and water to form an aqueous-based mixture containing an intermediate reaction product; separating the intermediate reaction product from the aqueous-based mixture by filtering out the suspended intermediate reaction product; and mixing the intermediate reaction product within a multi-acid solution containing sulfuric acid and nitric acid. Ammonium chloride is then added, thereby forming 2-nitroimino-5-nitrohexahydro-1,3,5-triazine. A gas generating system 200 containing a gas generant formed in accordance with the present invention is also contemplated.

### CROSS-REFERENCE TO RELATED APPLICATIONS

The present application claims the benefit of U.S. Provisional Application No. 60/762,839 having a filing date of Jan. 27, 2006.

### TECHNICAL FIELD

The present invention relates generally to gas generating systems, and to gas generant compositions employed in gas generator devices for automotive restraint systems, for example.

### BACKGROUND OF THE INVENTION

The present invention relates to nontoxic gas generating compositions that upon combustion rapidly generate gases that are useful for inflating occupant safety restraints in motor vehicles and specifically, the invention relates to thermally stable nonazide gas generants having not only acceptable burn rates, but that also, upon combustion, exhibit a relatively high gas volume to solid particulate ratio at acceptable flame temperatures.

The evolution from azide-based gas generants to nonazide gas generants is well-documented in the prior art. The advantages of nonazide gas generant compositions in comparison with azide gas generants have been extensively described in the patent literature, for example, U.S. Pat. Nos. 4,370,181; 4,909,549; 4,948,439; 5,084,118; 5,139,588 and 5,035,757, each patent hereby incorporated by reference.

In addition to a fuel constituent, pyrotechnic nonazide gas generants contain ingredients such as oxidizers to provide the required oxygen for rapid combustion and reduce the quantity of toxic gases generated, a catalyst to promote the conversion of toxic oxides of carbon and nitrogen to innocuous gases, and a slag forming constituent to cause the solid and liquid products formed during and immediately after combustion to agglomerate into filterable clinker-like particulates. Other optional additives, such as burning rate enhancers or ballistic modifiers and ignition aids, are used to control the ignitability and combustion properties of the gas generant.

One of the disadvantages of known nonazide gas generant compositions is the amount and physical nature of the solid residues formed during combustion. When employed in a vehicle occupant protection system, the solids produced as a result of combustion must be filtered and otherwise kept away from contact with the occupants of the vehicle. It is therefore highly desirable to develop compositions that produce a minimum of solid particulates while still providing adequate quantities of a nontoxic gas to inflate the safety device at a high rate.

The use of phase stabilized ammonium nitrate as an oxidizer, for example, is desirable because it generates abundant nontoxic gases and minimal solids upon combustion. To be useful, however, gas generants for automotive applications must be thermally stable when aged for 400 hours or more at 107.degree. C. The compositions must also retain structural integrity when cycled between −40.degree. C. and 107.degree. C. Further, gas generant compositions incorporating phase stabilized or pure ammonium nitrate sometimes exhibit poor thermal stability, and produce unacceptably high levels of toxic gases, CO and NOₓ for example, depending on the composition of the associated additives such as plasticizers and binders.

Yet another problem that must be addressed is that the U.S. Department of Transportation (DOT) regulations require “cap testing” for gas generants. Because of the sensitivity to detonation of fuels often used in conjunction with ammonium nitrate, many propellants incorporating ammonium nitrate do not pass the cap test unless shaped into large disks, which in turn reduces design flexibility of the inflator.

Accordingly, ongoing efforts in the design of automotive gas generating systems, for example, include other initiatives that desirably produce more gas and less solids without the drawbacks mentioned above. Simplification of the manufacture of gas generant constituents that produce little or no solids upon combustion is therefore also contemplated. U.S. Pat. No. 4,937,340, herein incorporated by reference, describes a solvent-based process for producing high-energy insensitive cyclic nitramines. Furthermore, extreme cooling is required to produce the nitramines described therein. Although the final product is desirable from a solids-producing standpoint, the extensive use of solvents increases the manufacturing cost due to environmental disposal and handling concerns. Reducing the amount of solvent in the manufacturing process would therefore be an advantage. Reducing the costs of manufacturing and increasing the product yield of a manufacturing process would also be an advantage. Other known processes require the use of 100% nitric acid for relatively high product yields, again with regard to nitramines such as 2-nitroimino-5-nitrohexahydro-1,3,5-triazine. For example, as published in Propellants, Explosives, Pyrotechnics 23, 179-181 (1998), herein incorporated by reference, although an 89% yield of 2-nitroimino-5-nitrohexahydro-1,3,5-triazine resulted from chloride-assisted nitrolysis of a tertiary amine, 100% nitric acid was required. This concentration is not available commercially because of its reactivity and hazardous nature. Accordingly, this concentrated acid must be manufactured on site if it is to be used. For these and other reasons, improvements in the art would be welcomed.

### SUMMARY OF THE INVENTION

The above-referenced concerns are resolved by gas generating systems including a gas generant composition containing 2-nitroimino-5-nitrohexahydro-1,3,5-triazine (NNT) as a fuel. More specifically, a gas generating system or vehicle occupant protection system contains NNT formed by an aqueous-based method described herein. Additionally, an aqueous-based method of forming NNT is described herein, wherein a dual-acid mixture combined with chloride-assisted nitrolysis of a tertiary amine, is employed. The reaction product of this method also presents a purer product.

### BRIEF DESCRIPTION OF THE DRAWINGS

FIG. 1 is an exemplary inflator incorporating a composition of the present invention.

FIG. 2 is an exemplary gas generating system, in this case a vehicle occupant protection system, incorporating the inflator of FIG. 1.

### DETAILED DESCRIPTION OF THE PREFERRED EMBODIMENT(S)

The present invention includes a water-based two-step method as illustrated below:

Step 1:

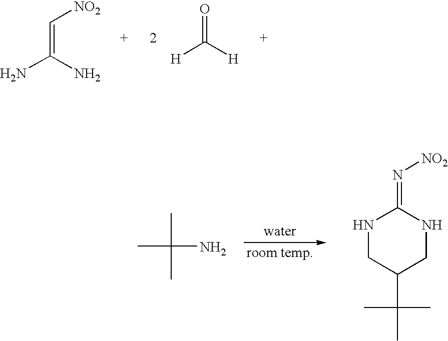

Step 2:

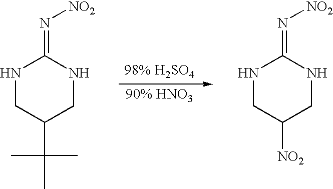

The new procedure substantially improves the desired product yield of Step 1 over known methods of production, and also substantially improves the desired product yield of Step 2 over known methods of production. In one embodiment, Step 1 may be carried out in water at room temperature for 48 hours to arrive at about 96% pure product, although less pure product may also result. In one embodiment, Step 2 is completed in a mixed acid system at 0 degrees Celsius to arrive at about 82% pure product, although less pure product may also result. The yields indicated provide an improved purity that substantially reduces the relative manufacturing cost of NNT.

In one process embodiment, the starting materials for the method illustrated include nitroguanidine, a 37 weight percent solution in water of formaldehyde, and a 98% pure solution of t-butyl amine. These are all dissolved or mixed in water at room temperature, as illustrated in Step 1. The reactants may for example be mixed in a known mixing vessel utilizing a planetary mixer over time.

Step 2 employs a mixture of 98% sulfuric acid and 90% fuming white nitric acid. The concentrations of the acid are preferably very concentrated and may be greater than that indicated. Further, a general volumetric ratio of sulfuric acid to nitric acid may be 25/75 to 75/25, wherein the volume of nitric acid is held constant. The intermediate reaction product of step 1 is dissolved in the acid and via chloride-assisted nitrolysis of the tert-butyl amine moiety, through the addition of ammonium chloride, forms 2-nitroimino-5-nitrohexahydro-1,3,5-triazine, as separated from the acidic solution. Combined with a relatively quick room temperature reaction in step 1, the present process provides a far more efficient method of production for NNT. It will be appreciated that other percent solutions than those indicated above may be employed in molar equivalent amounts that are consistent with the reaction provided. The molar equivalent ratio is two moles of formaldehyde per one mole of nitroguanidine and per one mole of t-butyl amine.

In yet another aspect of the invention, compositions formed in accordance with the present invention, may be employed within a gas generating system. For example, a vehicle occupant protection system made in a known way contains crash sensors (not shown) in electrical or operable communication with an airbag inflator in a steering wheel or otherwise within the vehicle, and also within a seatbelt assembly. The gas generating compositions formed in accordance with the present invention may be employed in both subassemblies within the broader vehicle occupant protection system or gas generating system. More specifically, each gas generator employed in the automotive gas generating system may contain a gas generating composition as described herein.

It should be noted that all percents given herein are weight percents based on the total weight of the gas generant composition. The chemicals described herein may be supplied by companies such as Aldrich Chemical Company and Polysciences, Inc. and Fisher Chemical for example.

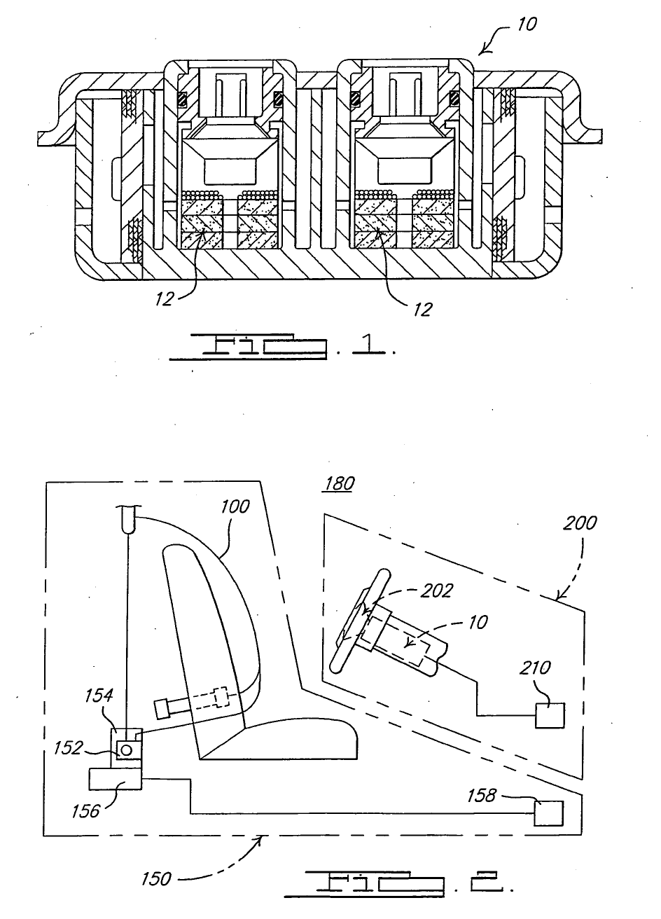

As shown in FIG. 1, an exemplary inflator incorporates a dual chamber design to tailor the force of deployment an associated airbag. In general, an inflator, containing a primary autoigniting gas generating composition 12 formed as described herein, may be manufactured as known in the art. U.S. Pat. Nos. 6,422,601, 6,805,377, 6,659,500, 6,749,219, and 6,752,421 exemplify typical airbag inflator designs and are each incorporated herein by reference in their entirety.

Referring now to FIG. 2, the exemplary inflator 10 described above may also be incorporated into a gas generating system such as an airbag or vehicle occupant protection system 200. Airbag system 200 includes at least one airbag 202 and an inflator 10 containing a gas generant composition 12 in accordance with the present invention, coupled to airbag 202 so as to enable fluid communication with an interior of the airbag. Airbag system 200 may also include (or be in communication with) a crash event sensor 210. Crash event sensor 210 includes a known crash sensor algorithm that signals actuation of airbag system 200 via, for example, activation of airbag inflator 10 in the event of a collision.

Referring again to FIG. 2, airbag system 200 may also be incorporated into a broader, more comprehensive vehicle occupant restraint system 180 including additional elements such as a safety belt assembly 150. FIG. 2 shows a schematic diagram of one exemplary embodiment of such a restraint system. Safety belt assembly 150 includes a safety belt housing 152 and a safety belt 100 extending from housing 152. A safety belt retractor mechanism 154 (for example, a spring-loaded mechanism) may be coupled to an end portion of the belt. In addition, a safety belt pretensioner 156 containing propellant 12 and autoignition 14 may be coupled to belt retractor mechanism 154 to actuate the retractor mechanism in the event of a collision. Typical seat belt retractor mechanisms which may be used in conjunction with the safety belt embodiments of the present invention are described in U.S. Pat. Nos. 5,743,480, 5,553,803, 5,667,161, 5,451,008, 4,558,832 and 4,597,546, incorporated herein by reference. Illustrative examples of typical pretensioners with which the safety belt embodiments of the present invention may be combined are described in U.S. Pat. Nos. 6,505,790 and 6,419,177, incorporated herein by reference.

Safety belt assembly 150 may also include (or be in operable communication with) a crash event sensor 158 (for example, an inertia sensor or an accelerometer, not shown) including a known crash sensor algorithm that signals actuation of belt pretensioner 156 via, for example, activation of a pyrotechnic igniter (not shown) incorporated into the pretensioner. U.S. Pat. Nos. 6,505,790 and 6,419,177, previously incorporated herein by reference, provide illustrative examples of pretensioners actuated in such a manner.

It should be appreciated that safety belt assembly 150, airbag system 200, and more broadly, vehicle occupant protection system 180 exemplify but do not limit gas generating systems contemplated in accordance with the present invention.

Compositions may be formed using 2-nitroimino-5-nitrohexahydro-1,3,5-triazine (NNT) as a fuel at about 20-50 wt % of the total composition, and as formed in the present invention. Furthermore, other constituents typically contained in gas generant compositions may be mixed with NNT including an oxidizer provided at about 50-80 wt %. The primary oxidizer is selected from metal and nonmetal salts of chlorates, perchlorates, nitrates, and nitrites including potassium perchlorate, ammonium perchlorate, strontium nitrate, potassium nitrate, ammonium nitrate, and phase stabilized ammonium nitrate (PSAN) (stabilized with 10-15 wt % potassium nitrate relative to the total weight of the PSAN, for example); basic metal nitrates such as basic copper nitrate; basic metal carbonates; metal oxides such as iron oxide, di-potassium oxide, and potassium oxide; and transitional metal complexes including nitrates and nitrites such as Cu(NH₃)₂NO₂.

A secondary fuel, provided at no more than 20% by weight, may be selected from known fuels such as azoles including tetrazoles and triazoles, and guanidines. Other additives may include graphite, silicas, clays, talcs, micas, and other slag formers, combustion modifiers, and processing aids as known in the art. U.S. Pat. No. 5,035,757 exemplifies some of these fuels and is herein incorporated by reference.

An exemplary composition contains 35.25% NNT oxygen balanced with 65.75% PSAN (stabilized with 10% potassium nitrate relative to the total weight of the PSAN). The compositions preferably contain 50-80% PSAN and 20-50% NNT. If secondary oxidizers are employed, they are employed at no more than 20 wt % of the total composition, and the total oxygen balance is tailored to maximize the formation of combustion products such as carbon dioxide and water. It should be appreciated that unless otherwise indicated, all percentages stated with regard to the composition are stated as weight percents relative to the total weight of the gas generant composition.

The compositions may be dry or wet mixed using methods known in the art. The various constituents may be generally provided in particulate form and mixed to form a uniform mixture with the other gas generant constituents. The mixture is then pelletized or formed into other useful shapes in a safe manner known in the art.

### Exemplary Method

### Steps 1: Formation of an Intermediate Reaction Product

1. Nitroguanidine was milled for 15 minutes in the M-18-5 mill prior to use.
2. Nitroguanidine was added to a mixing vessel containing a strong mechanical mixer, along with water (750 ml), 37% formaldehyde solution in water (215 ml), and ammonium lauryl sulfate (2 drops). It has been found that the use of this soap or other soaps, or phase transfer catalyst, provides for enhanced reaction between phases.
3. T-butyl amine (155 ml, 98%) was then added slowly. A slight exotherm was noted. With larger batches, a cooling jacket is recommended about the mixer to maintain a temperature reasonably close to room temperature. The mixture, although continually stirred, became relatively more viscous with the addition of the t-butyl amine. The mixture presented a white slurry.
4. With continual stirring, reaction proceeded at room temperature for about 24-48 hours, with 48 hours being recommended. The mixture presented a white slurry at the end of the reaction period. As determined by DSC analysis, a percent yield of about 97.5% was realized.
5. The mixer vessel contents were then filtered and washed with four aliquots of 250 ml of water, followed by two aliquots of 100 ml of acetone. A filter cake was then removed from the filter funnel and air-dried in a hood overnight, or for about 6-8 hours. Care should be taken not to heat the material while drying due to potential decomposition while wet. The filtrate or intermediate reaction product (before washing) may, if desired, be recycled and recharged with more nitroguanidine, formaldehyde, and t-butyl amine.

### Steps 2: Formation of 2-nitroimino-5-nitrohexahydro-1,3,5-triazine

6. A 50/50 solution of sulfuric acid and nitric acid was prepared by dissolving 98% sulfuric acid (500 ml) into 90% white fuming nitric acid (500 ml) which has been pre-chilled to 0 degrees Celsius in an ice bath, within a mixing vessel. It should be emphasized that the nitric acid may also be characterized as any commercially available form of nitric acid, exhibiting 90% or greater molarity.
7. Once the mixture has stabilized to a temperature of 0 degrees Celsius, the intermediate reaction product is added in portions until it completely dissolves. Care should be taken to ensure consistently rapid stirring along with managed heat control. Again, a cooling jacket may be employed about the mixing vessel to maintain the temperature about room temperature.
8. Ammonium chloride (48 grams) was then added slowly. In practice, about one mol of ammonium chloride should be added for each mol of intermediate reaction product. Some foaming will occur, which is best managed with efficient stirring and an inert gas purge. The solution was kept cold during the entire addition with the addition of ice to the mixing vessel (or with activation of the cooling jacket), and was permitted to slowly warm to room temperature overnight. If a cooling jacket is employed, the jacket is deactivated after the addition of the ammonium chloride is complete.
9. The acidic solution was then quenched by slowly pouring into four gallons of ice and water (about ⅔ ice) with consistent and strong stirring. After several minutes a slightly yellow/green precipitate was formed, and then filtered off.
10. The filtrate was then washed with copious amounts of water, about six aliquots of 250 ml each to remove the acid. The filter cake was then rinsed with 200 ml of acetone to remove impurities.
11. The slightly green filter cake was then dried overnight in the hood at room temperature. The resultant solid was confirmed by IR analysis to be 2-nitroimino-5-nitrohexahydro-1,3,5-triazine, and presented a 97.5% product yield.
12. If desired, the resulting amorphous material may be purified even further by recrystallization in distilled water heated to a boil, to totally dissolve the solid. The solution is then cooled to precipitate a purer solid, thereby resulting in white cubic crystals with minimal product loss (about 0.5%). Thus shape presents a more flowable geometry for processing into gas generating compositions.

The present description is for illustrative purposes only, and should not be construed to limit the breadth of the present invention in any way. Thus, those skilled in the art will appreciate that various modifications could be made to the presently disclosed embodiments without departing from the scope of the present invention as defined in the appended claims.

1. A method of forming 2-nitroimino-5-nitrohexahydro-1,3,5-triazine comprising the steps of:
mixing nitroguanidine, formaldehyde, tent-butyl amine and water to form an aqueous-based mixture containing an intermediate reaction product;
separating the intermediate reaction product from the aqueous-based mixture by filtering out the intermediate reaction product;
mixing the intermediate reaction product within a multi-acid solution containing sulfuric acid and nitric acid, thoroughly dissolving the intermediate reaction product; and
adding ammonium chloride to the mixture, thereby forming 2-nitroimino-5-nitrohexahydro-1,3,5-triazine.

2. A method of forming 2-nitroimino-5-nitrohexahydro-1,3,5-triazine comprising the steps of:
mixing about one mol nitroguanidine and about two cools formaldehyde in a solvent effective amount of water, to form a first mixture;
adding about one mol of tert-butyl amine to the first mixture, and continuing to mix the mixture to form a white slurry;
filtering the white slurry to yield a first solid;
washing the first solid;
drying the first solid;
mixing the solid to a multi-acid solution containing sulfuric acid and nitric acid to form a second mixture;
adding ammonium chloride to the second mixture and mixing, while simultaneously cooling the second mixture; and
adding ice water to the second mixture and continuing to mix, until a second solid forms.

3. The method of claim 2 further comprising the step of adding a soap to the first mixture prior to mixing in the tert-butyl amine.

4. The method of claim 3 wherein said soap is ammonium lauryl sulfate.

5. The method of claim 2 further comprising the step of simultaneously cooling the first mixture as the tert-butyl amine is added, thereby maintaining the temperature of the first mixture to about room temperature.

6. The method of claim 2 wherein the step of washing the first solid further comprises washing the first solid with water and then washing the first solid with acetone.

7. A gas generant composition containing the second solid of claim 2.

8. A gas generant fuel formed by the method comprising the steps of:
mixing about one mol nitroguanidine and about two mols formaldehyde in a solvent effective amount of water, to form a first mixture;
adding about one mol of tert-butyl amine to the first mixture, and continuing to mix the mixture to form a white slurry;
filtering the white slurry to yield a first solid;
washing the first solid;
drying the first solid;
mixing the solid to a multi-acid solution containing sulfuric acid and nitric acid to form a second mixture;
adding ammonium chloride to the second mixture and mixing, while simultaneously cooling the second mixture; and
adding ice water to the second mixture and continuing to mix, until a second solid forms.

9. A gas generant composition comprising:
a fuel of claim 8; and
an oxidizer,
wherein said fuel is provided at about 20-50 weight percent of the total composition.

10. The gas generant composition of claim 9 wherein said oxidizer is phase stabilized ammonium nitrate provided at about 50-80 weight percent of the total composition.

11. The gas generant composition of claim 9 wherein said oxidizer is selected from metal and nonmetal salts of chlorates, perchlorates, nitrates, and nitrites; basic metal nitrates; basic metal carbonates; metal oxides; and transitional metal complexes of nitrates and nitrites.

12. The gas generant composition of claim 9 wherein said oxidizer is selected from potassium perchlorate, ammonium perchlorate, strontium nitrate, potassium nitrate, ammonium nitrate, phase stabilized ammonium nitrate, basic copper nitrate, iron oxide, di-potassium oxide, and potassium oxide.

13. The gas generant composition of claim 9 further comprising a secondary fuel selected from azoles, tetrazoles, triazoles, and guanidines, said secondary fuel provided at no more than 20 weight percent of the total composition.

14. The gas generant composition of claim 9 further comprising an additive selected from graphite, silicas, clays, talcs, and micas.

15. The gas generant composition of claim 9 further comprising a slag former.

16. The gas generant composition of claim 9 further comprising a combustion modifier.

17. The gas generant composition of claim 9 further comprising a processing aid.

18. The method of claim 2 further comprising the steps of:
filtering the second solid from the second mixture;
washing the second solid; and
air-drying the second solid.

19. The method of claim 18 further comprising the steps of:
dissolving the second solid in heated distilled water to form a third mixture; and
cooling the third mixture to form a third solid.

20. The method of claim 2 wherein said multi-acid solution is 90% fuming white nitric acid and 98% sulfuric acid, the nitric acid and the sulfuric acid each provided at about 25-75% by volume of the multi-acid solution.
